(19) United States
(12) Patent Application Publication
(10) Pub. No.: US 2009/0246174 A1
(43) Pub. Date: Oct. 1, 2009

(54) TREATMENT FOR CUTANEOUS T CELL LYMPHOMA

(76) Inventors: Alain H. Rook, Wynnewood, PA (US); Bernice M. Benoit, Prospect Park, PA (US); Maria Wysocka, Drexel Hill, PA (US); Sarah M. Bray, Pittsburgh, PA (US); Richard L. Miller, Maplewood, MN (US); Mark A. Tomai, Woodbury, MN (US)

Correspondence Address: 3M INNOVATIVE PROPERTIES COMPANY, PO BOX 33427, ST. PAUL, MN, 55133-3427, US

(21) Appl. No.: 12/159,058

(22) PCT Filed: Dec. 28, 2006
(86) PCT No.: PCT/US06/49525

§ 371 Date: Oct. 8, 2008

Related U.S. Application Data
(60) Provisional application No. 60/754,476, filed on Dec. 28, 2005.

(51) Int. Cl. A61K 38/21; C12N 5/06; A61K 31/4745

(52) U.S. Cl. 424/85.5; 435/375; 514/293; 424/85.7

(57) ABSTRACT

The present invention provides method for treating a patient with cutaneous T cell lymphoma (CTCL). Generally, the methods include administering to the patient an IRM compound in an amount effective to ameliorate at least one symptom or clinical sign of CTCL. In some embodiments, the methods also include administering to the patient a priming dose of a Type I interferon. In another aspect, the invention provides methods of increasing a cell-mediated immune response of a cell population that includes cells affected by cutaneous T cell lymphoma. Generally, the methods include contacting the cell population with an IRM compound in an amount effective to increase at least one cell-mediated immune activity of the cell population. In some embodiments, the methods include contacting the cell population with a priming dose of a Type I interferon.

### BACKGROUND

There has been a major effort in recent years, with significant success, to discover new drug compounds that act by stimulating certain key aspects of the immune system, as well as by suppressing certain other aspects (see, e.g., U.S. Pat. Nos. 6,039,969 and 6,200,592). These compounds, referred to herein as immune response modifiers (IRMs), appear to act through basic immune system mechanisms known as Toll-like receptors (TLRs) to induce selected cytokine biosynthesis, induction of co-stimulatory molecules, and increased antigen-presenting capacity.

They may be useful for treating a wide variety of diseases and conditions. For example, certain IRMs may be useful for treating viral diseases (e.g., human papilloma virus, hepatitis, herpes), neoplasias (e.g., basal cell carcinoma, squamous cell carcinoma, actinic keratosis, melanoma), and T_{H}2-mediated diseases (e.g., asthma, allergic rhinitis, atopic dermatitis), auto-immune diseases (e.g., multiple sclerosis), and are also useful as vaccine adjuvants.

Many of the IRM compounds are small organic molecule imidazoquinoline amine derivatives (see, e.g., U.S. Pat. No. 4,689,338), but a number of other compound classes are known as well (see, e.g., U.S. Pat. Nos. 5,446,153; 6,194,425; and 6,110,929; and International Publication Number WO 2005/079195) and more are still being discovered. Other IRMs have higher molecular weights, such as oligonucleotides, including CpGs (see, e.g., U.S. Pat. No. 6,194,388).

In view of the great therapeutic potential for IRMs, and despite the important work that has already been done, there is a substantial ongoing need to expand their uses and therapeutic benefits.

### SUMMARY

It has been found that certain small molecule IRMs can be used in the treatment of cutaneous T cell lymphoma (CTCL).

Accordingly, the present invention provides a method of increasing a cell-mediated immune response of a cell population that includes cells affected by cutaneous T cell lymphoma. In some embodiments, the method generally includes contacting the cell population with an immune response modifier (IRM) compound in an amount effective to increase at least one cell-mediated immune activity of the cell population, wherein the IRM compound is other than 1-(2-methylpropyl)-1H-imidazo[4,5-c]quinolin-4-amine. In other embodiments, the method generally includes contacting the cell population with an IRM compound in an amount effective to increase at least one cell-mediated immune activity of the cell population, wherein the IRM compound is a substituted imidazoquinoline amine, a tetrahydroimidazoquinoline amine, an imidazopyridine amine, a 1,2-bridged imidazoquinoline amine, a 6,7-fused cycloalkylimidazopyridine amine, an imidazonaphthyridine amine, a tetrahydroimidazonaphthyridine amine, an oxazoloquinoline amine, a thiazoloquinoline amine, an oxazolopyridine amine, a thiazolopyridine amine, an oxazolonaphthyridine amine, a thiazolonaphthyridine amine, a pyrazolopyridine amine, a pyrazoloquinoline amine, a tetrahydropyrazoloquinoline amine, a pyrazolonaphthyridine amine, or a tetrahydropyrazolonaphthyridine amine. In still other embodiments, the method includes contacting the cell population with a priming dose of either IFN-α or IFN-γ, and then contacting the cell population with an IRM compound in an amount effective to increase at least one cell-mediated immune activity of the cell population.

In another aspect, the present invention also provides a method of treating a patient with cutaneous T cell lymphoma (CTCL). In some embodiments, the method generally includes administering to a CTCL patient an amount of a pharmaceutical composition comprising an IRM compound effective for ameliorating at least one symptom or clinical sign of cutaneous T cell lymphoma, wherein the IRM compound is other than 1-(2-methylpropyl)-1H-imidazo[4,5-c]quinolin-4-amine. In other embodiments, the method generally includes administering to a CTCL patient an amount of a pharmaceutical composition comprising an IRM compound effective for ameliorating at least one symptom or clinical sign of cutaneous T cell lymphoma, wherein the IRM compound is a substituted imidazoquinoline amine, a tetrahydroimidazoquinoline amine, an imidazopyridine amine, a 1,2-bridged imidazoquinoline amine, a 6,7-fused cycloalkylimidazopyridine amine, an imidazonaphthyridine amine, a tetrahydroimidazonaphthyridine amine, an oxazoloquinoline amine, a thiazoloquinoline amine, an oxazolopyridine amine, a thiazolopyridine amine, an oxazolonaphthyridine amine, a thiazolonaphthyridine amine, a pyrazolopyridine amine, a pyrazoloquinoline amine, a tetrahydropyrazoloquinoline amine, a pyrazolonaphthyridine amine, or a tetrahydropyrazolonaphthyridine amine. In still other embodiments, the method includes administering to the patient a priming dose of either IFN-α or IFN-γ, and then administering to the patient an IRM compound in an amount effective for ameliorating at least one symptom or clinical sign of cutaneous T cell lymphoma.

Various other features and advantages of the present invention should become readily apparent with reference to the following detailed description, examples, claims and appended drawings. In several places throughout the specification, guidance is provided through lists of examples. In each instance, the recited list serves only as a representative group and should not be interpreted as an exclusive list.

### BRIEF DESCRIPTION OF THE DRAWINGS

FIG. 1 is a bar graph showing cytokine production by PBMCs from CTCL patients in response to IRM compounds.

FIG. 2 is a bar graph showing activation of NK cells from CTCL patients in response to IRM compounds.

FIG. 3 is a line graph showing cytolytic activity of NK cells from CTCL patients in response to IRM compounds.

FIG. 4A is a bar graph showing enhancement of IL-12 production by PBMCs from a CTCL patient in response to IRM compounds when primed with IFN-γ.

FIG. 4B is a bar graph showing enhancement of IL-12 production by PBMCs from a second CTCL patient in response to IRM compounds when primed with IFN-γ.

### DETAILED DESCRIPTION OF ILLUSTRATIVE EMBODIMENTS OF THE INVENTION

The present invention provides a method of treating cutaneous T cell lymphoma (CTCL). Patients with advanced CTCL have a significantly impaired ability to generate a cell-mediated immune response, at least in part because they have abnormally low numbers of dendritic cells (DCs), cells that play an important role in cell-mediated immunity. The impaired cell-mediated immune response makes it difficult for the patient's immune system to control and contain the CTCL disease. The invention uses immune response modifier (IRM) compounds to stimulate immune responses by other, still responsive immune cell populations to help control and contain the CTCL disease.

As used herein, the following terms shall have the indicated meanings:

“Agonist” refers to a compound that can combine with a receptor (e.g., a TLR) to induce a cellular activity. An agonist may be a ligand that directly binds to the receptor. Alternatively, an agonist may combine with a receptor indirectly by, for example, (a) forming a complex with another molecule that directly binds to the receptor, or (b) otherwise results in the modification of another compound so that the other compound directly binds to the receptor. An agonist may be referred to as an agonist of a particular TLR (e.g., a TLR6 agonist) or a particular combination of TLRs (e.g., a TLR 7/8 agonist—an agonist of both TLR7 and TLR8).

“Ameliorate” refers to any reduction in the extent, severity, frequency, and/or likelihood of a symptom or clinical sign characteristic of a particular condition.

“Cell-mediated immune activity” refers to a biological activity considered part of a cell-mediated immune response such as, for example, an increase in the production of at least one T_{H}1 cytokine.

“Immune cell” refers to cell of the immune system, i.e., a cell directly or indirectly involved in the generation or maintenance of an immune response, whether the immune response is innate, acquired, humoral, or cell-mediated.

“Sign” or “clinical sign” refers to an objective physical finding relating to a particular condition capable of being found by one other than the patient.

“Symptom” refers to any subjective evidence of disease or of a patient's condition.

“Treat” or variations thereof refer to reducing, limiting progression, ameliorating, or resolving, to any extent, the symptoms or signs related to a condition.

As used herein, “a,” “an,” “the,” “at least one,” and “one or more” are used interchangeably. Thus, for example, a pharmaceutical composition comprising “an” IRM compound can be interpreted to mean that the pharmaceutical composition includes at least one IRM compound.

Also herein, the recitations of numerical ranges by endpoints include all numbers subsumed within that range (e.g., 1 to 5 includes 1, 1.5, 2, 2.75, 3, 3.80, 4, 5, etc.).

Cutaneous T-cell lymphoma (CTCL) is a relatively rare disease, with an annual incidence of about 0.29 cases per 100,000 persons in the United States. It is about half as common in Eastern Europe. However, this discrepancy may be attributed to a differing physician awareness of the disease rather than a true difference in occurrence. In the Unites States, there are about 500-600 new cases a year and about 100-200 deaths. CTCL is usually seen in older adults; the median age at diagnosis is 55-60 years. It strikes twice as many men as women. The average life expectancy at diagnosis is 7-10 years, even without treatment.

CTCL is an indolent (low grade) cancer of the white blood cells that primarily affects the skin and only secondarily affects other sites. This disease involves the uncontrollable proliferation of T lymphocytes known as helper T (T_{H}) cells. The proliferation of helper T cells results in the penetration, or infiltration, of these abnormal cells into the epidermal layer of the skin. The skin may react with itchy, slightly scaling lesions, although the sites of greatest infiltration do not necessarily correspond to the sites of the lesions. The lesions are most often located on the trunk, but can be present on any part of the body. In the most common course of the disease, also known as mycosis fungoides (MF), the patchy lesions progress to palpable plaques that are deeper red and have more defined edges. Eventually, skin tumors may develop. Finally, the cancer may progress to extracutanous involvement, often in the lymph nodes or the viscera. In rare cases, affected individuals may develop Sezary syndrome (SS), a leukemic variant of mycosis fungoides.

The proliferative T lymphocytes of CTCL are characterized by the phenotype CD4⁺/CD45R⁺/CLA⁺/CCR4⁺. Mycosis fungoides and Sezary syndrome differ in the involvement of the peripheral blood: MF typically appears without overt involvement of the peripheral blood by circulating malignant T cells, whereas Sezary syndrome typically includes malignant T cells disseminated into the blood stream. Involvement of the peripheral blood is typically associated with a decrease in cell-mediated immunity including a decrease in the production of T_{H}1-type cytokines such as, for example, IFN-γ and IL-2, and increased production of T_{H}2-type cytokines such as, for example, IL-4 and IL-5.

Exogenous administration of T_{H}1-type cytokines produces measurable clinical responses in treated patients. For example, administration of IFN-α, IFN-γ, and/or IL-12 have been used in such therapies, but identification of effective therapeutic agents with a low occurrence of side effects and an ability to stimulate multiple components of the immune system continues.

Immune response modifiers (“IRMs”) include compounds that possess potent immunomodulating activity including but not limited to antiviral and antitumor activity. Certain IRMs modulate the production and secretion of cytokines. For example, certain IRM compounds induce the production and secretion of cytokines such as, e.g., Type I interferons, TNF-α, IL-1, IL-6, IL-8, IL-0, IL-12, MIP-1, and/or MCP-1. As another example, certain IRM compounds can inhibit production and secretion of certain T_{H}2 cytokines, such as IL-4 and IL-5. Additionally, some IRM compounds are said to suppress IL-1 and TNF (U.S. Pat. No. 6,518,265).

Certain IRMs are small organic molecules (e.g., molecular weight under about 1000 Daltons, preferably under about 500 Daltons, as opposed to large biological molecules such as proteins, peptides, nucleic acids, and the like) such as those disclosed in, for example, U.S. Pat. Nos. 4,689,338; 4,929,624; 5,266,575; 5,268,376; 5,346,905; 5,352,784; 5,389,640; 5,446,153; 5,482,936; 5,756,747; 6,110,929; 6,194,425; 6,331,539; 6,376,669; 6,451,810; 6,525,064; 6,541,485; 6,545,016; 6,545,017; 6,573,273; 6,656,938; 6,660,735; 6,660,747; 6,664,260; 6,664,264; 6,664,265; 6,667,312; 6,670,372; 6,677,347; 6,677,348; 6,677,349; 6,683,088; 6,756,382; 6,797,718; 6,818,650; and 7,7091,214; U.S. Patent Publication Nos. 2004/0091491; 2004/0176367; and 2006/0100229; and International Publication Nos. WO 2005/18551, WO 2005/18556, WO 2005/20999, WO 2005/032484, WO 2005/048933, WO 2005/048945, WO 2005/051317, WO 2005/051324, WO 2005/066169, WO 2005/066170, WO 2005/066172, WO 2005/076783, WO 2005/079195, WO 2005/094531, WO 2005/123079, WO 2005/123080, WO 2006/009826, WO 2006/009832, WO 2006/026760, WO 2006/028451, WO 2006/028545, WO 2006/028962, WO 2006/029115, WO 2006/038923, WO 2006/065280, WO 2006/074003, WO 2006/083440, WO 2006/086449, WO 2006/091394, WO 2006/086633, WO 2006/086634, WO 2006/091567, WO 2006/091568, WO 2006/091647, WO 2006/093514, and WO 2006/098852.

Additional examples of small molecule IRMs include certain purine derivatives (such as those described in U.S. Pat. Nos. 6,376,501, and 6,028,076), certain imidazoquinoline amide derivatives (such as those described in U.S. Pat. No. 6,069,149), certain imidazopyridine derivatives (such as those described in U.S. Pat. No. 6,518,265), certain benzimidazole derivatives (such as those described in U.S. Pat. No. 6,387,938), certain derivatives of a 4-aminopyrimidine fused to a five membered nitrogen containing heterocyclic ring (such as adenine derivatives described in U.S. Pat. Nos. 6,376,501; 6,028,076 and 6,329,381; and in WO 02/08905), and certain 3-β-D-ribofuranosylthiazolo[4,5-d]pyrimidine derivatives (such as those described in U.S. Publication No. 2003/0199461), and certain small molecule immuno-potentiator compounds such as those described, for example, in U.S. Patent Publication No. 2005/0136065.

Other IRMs include large biological molecules such as oligonucleotide sequences. Some IRM oligonucleotide sequences contain cytosine-guanine dinucleotides (CpG) and are described, for example, in U.S. Pat. Nos. 6,194,388; 6,207,646; 6,239,116, 6,339,068; and 6,406,705. Some CpG-containing oligonucleotides can include synthetic immunomodulatory structural motifs such as those described, for example, in U.S. Pat. Nos. 6,426,334 and 6,476,000. Other IRM nucleotide sequences lack CpG sequences and are described, for example, in International Patent Publication No. WO 00/75304. Still other IRM nucleotide sequences include guanosine- and uridine-rich single-stranded RNA (ssRNA) such as those described, for example, in Heil et al., Science, vol. 303, pp. 1526-1529, Mar. 5, 2004.

Other IRMs include biological molecules such as aminoalkyl glucosaminide phosphates (AGPs) and are described, for example, in U.S. Pat. Nos. 6,113,918; 6,303,347; 6,525,028; and 6,649,172.

Unless otherwise indicated, reference to a compound can include the compound in any pharmaceutically acceptable form, including any isomer (e.g., diastereomer or enantiomer), salt, solvate, polymorph, and the like. In particular, if a compound is optically active, reference to the compound can include each of the compound's enantiomers as well as racemic mixtures of the enantiomers.

In some embodiments of the present invention, the IRM compound may be an agonist of at least one TLR such as, for example, TLR7 or TLR8. The IRM may also in some cases be an agonist of TLR 9. In some embodiments, the IRM compound may be an agonist of at least one of TLR7 and TLR8 such as, for example, a TLR7/8 agonist, a TLR8-selective agonist, or a TLR7-selective agonist. As used herein, the term “TLR8-selective agonist” refers to any compound that acts as an agonist of TLR8, but does not act as an agonist of TLR7. A “TLR7-selective agonist” refers to a compound that acts as an agonist of TLR7, but does not act as an agonist of TLR8. A “TLR7/8 agonist” refers to a compound that acts as an agonist of both TLR7 and TLR8.

A TLR8-selective agonist or a TLR7-selective agonist may act as an agonist for the indicated TLR and one or more of TLR1, TLR2, TLR3, TLR4, TLR5, TLR6, TLR9, or TLRL0. Accordingly, while “TLR8-selective agonist” may refer to a compound that acts as an agonist for TLR8 and for no other TLR, it may alternatively refer to a compound that acts as an agonist of TLR8 and, for example, TLR6. Similarly, “TLR7-selective agonist” may refer to a compound that acts as an agonist for TLR7 and for no other TLR, but it may alternatively refer to a compound that acts as an agonist of TLR7 and, for example, TLR6.

The TLR agonism for a particular compound may be assessed in any suitable manner. For example, assays and recombinant cell lines suitable for detecting TLR agonism of test compounds are described, for example, in U.S. Patent Publication Nos. US2004/0014779, US2004/0132079, US2004/0162309, US2004/0171086, US2004/0191833, and US2004/0197865.

Regardless of the particular assay employed, a compound can be identified as an agonist of a particular TLR if performing the assay with a compound results in at least a threshold increase of some biological activity mediated by the particular TLR. Conversely, a compound may be identified as not acting as an agonist of a specified TLR if, when used to perform an assay designed to detect biological activity mediated by the specified TLR, the compound fails to elicit a threshold increase in the biological activity. Unless otherwise indicated, an increase in biological activity refers to an increase in the same biological activity over that observed in an appropriate control. An assay may or may not be performed in conjunction with the appropriate control. With experience, one skilled in the art may develop sufficient familiarity with a particular assay (e.g., the range of values observed in an appropriate control under specific assay conditions) that performing a control may not always be necessary to determine the TLR agonism of a compound in a particular assay.

The precise threshold increase of TLR-mediated biological activity for determining whether a particular compound is or is not an agonist of a particular TLR in a given assay may vary according to factors known in the art including but not limited to the biological activity observed as the endpoint of the assay, the method used to measure or detect the endpoint of the assay, the signal-to-noise ratio of the assay, the precision of the assay, and whether the same assay is being used to determine the agonism of a compound for more than one TLR. Accordingly it is not practical to set forth generally the threshold increase of TLR-mediated biological activity required to identify a compound as being an agonist or a non-agonist of a particular TLR for all possible assays. Those of ordinary skill in the art, however, can readily determine the appropriate threshold with due consideration of such factors.

Assays employing HEK293 cells transfected with an expressible TLR structural gene may use a threshold of, for example, at least a three-fold increase in a TLR-mediated biological activity (e.g., NFκB activation) when the compound is provided at a concentration of, for example, from about 1 μM to about 10 μM for identifying a compound as an agonist of the TLR transfected into the cell. However, different thresholds and/or different concentration ranges may be suitable in certain circumstances. Also, different thresholds may be appropriate for different assays.

In some embodiments of the present invention, the IRM compound may be a small molecule immune response modifier (e.g., molecular weight of less than about 1000 Daltons).

In some embodiments of the present invention, the IRM compound may include a 2-aminopyridine fused to a five membered nitrogen-containing heterocyclic ring, or a 4-aminopyrimidine fused to a five membered nitrogen-containing heterocyclic ring. Compounds having a 2-aminopyridine fused to a five membered nitrogen-containing heterocyclic ring suitable for practicing the invention include, for example, imidazoquinoline amines including but not limited to substituted imidazoquinoline amines such as, for example, amide substituted imidazoquinoline amines, sulfonamide substituted imidazoquinoline amines, urea substituted imidazoquinoline amines, aryl ether substituted imidazoquinoline amines, heterocyclic ether substituted imidazoquinoline amines, amido ether substituted imidazoquinoline amines, sulfonamido ether substituted imidazoquinoline amines, urea substituted imidazoquinoline ethers, thioether substituted imidazoquinoline amines, hydroxylamine substituted imidazoquinoline amines, oxime substituted imidazoquinoline amines, 6-, 7-, 8-, or 9-aryl, heteroaryl, aryloxy or arylalkyleneoxy substituted imidazoquinoline amines, and imidazoquinoline diamines; tetrahydroimidazoquinoline amines including but not limited to amide substituted tetrahydroimidazoquinoline amines, sulfonamide substituted tetrahydroimidazoquinoline amines, urea substituted tetrahydroimidazoquinoline amines, aryl ether substituted tetrahydroimidazoquinoline amines, heterocyclic ether substituted tetrahydroimidazoquinoline amines, amido ether substituted tetrahydroimidazoquinoline amines, sulfonamido ether substituted tetrahydroimidazoquinoline amines, urea substituted tetrahydroimidazoquinoline ethers, thioether substituted tetrahydroimidazoquinoline amines, hydroxylamine substituted tetrahydroimidazoquinoline amines, oxime substituted tetrahydroimidazoquinoline amines, and tetrahydroimidazoquinoline diamines; imidazopyridine amines including but not limited to amide substituted imidazopyridine amines, sulfonamide substituted imidazopyridine amines, urea substituted imidazopyridine amines, aryl ether substituted imidazopyridine amines, heterocyclic ether substituted imidazopyridine amines, amido ether substituted imidazopyridine amines, sulfonamido ether substituted imidazopyridine amines, urea substituted imidazopyridine ethers, and thioether substituted imidazopyridine amines; 1,2-bridged imidazoquinoline amines; 6,7-fused cycloalkylimidazopyridine amines; imidazonaphthyridine amines; tetrahydroimidazonaphthyridine amines; oxazoloquinoline amines; thiazoloquinoline amines; oxazolopyridine amines; thiazolopyridine amines; oxazolonaphthyridine amines; thiazolonaphthyridine amines; pyrazolopyridine amines; pyrazoloquinoline amines; tetrahydropyrazoloquinoline amines; pyrazolonaphthyridine amines; tetrahydropyrazolonaphthyridine amines; and 1H-imidazo dimers fused to pyridine amines, quinoline amines, tetrahydroquinoline amines, naphthyridine amines, or tetrahydronaphthyridine amines.

In certain embodiments, the IRM compound may be an imidazonaphthyridine amine, a tetrahydroimidazonaphthyridine amine, an oxazoloquinoline amine, a thiazoloquinoline amine, an oxazolopyridine amine, a thiazolopyridine amine, an oxazolonaphthyridine amine, a thiazolonaphthyridine amine, a pyrazolopyridine amine, a pyrazoloquinoline amine, a tetrahydropyrazoloquinoline amine, a pyrazolonaphthyridine amine, or a tetrahydropyrazolonaphthyridine amine.

In certain embodiments, the IRM compound may be a substituted imidazoquinoline amine, a tetrahydroimidazoquinoline amine, an imidazopyridine amine, a 1,2-bridged imidazoquinoline amine, a 6,7-fused cycloalkylimidazopyridine amine, an imidazonaphthyridine amine, a tetrahydroimidazonaphthyridine amine, an oxazoloquinoline amine, a thiazoloquinoline amine, an oxazolopyridine amine, a thiazolopyridine amine, an oxazolonaphthyridine amine, a thiazolonaphthyridine amine, a pyrazolopyridine amine, a pyrazoloquinoline amine, a tetrahydropyrazoloquinoline amine, a pyrazolonaphthyridine amine, or a tetrahydropyrazolonaphthyridine amine.

As used herein, a substituted imidazoquinoline amine refers to an amide substituted imidazoquinoline amine, a sulfonamide substituted imidazoquinoline amine, a urea substituted imidazoquinoline amine, an aryl ether substituted imidazoquinoline amine, a heterocyclic ether substituted imidazoquinoline amine, an amido ether substituted imidazoquinoline amine, a sulfonamido ether substituted imidazoquinoline amine, a urea substituted imidazoquinoline ether, a thioether substituted imidazoquinoline amine, a hydroxylamine substituted imidazoquinoline amine, an oxime substituted imidazoquinoline amine, a 6-, 7-, 8-, or 9-aryl, heteroaryl, aryloxy or arylalkyleneoxy substituted imidazoquinoline amine, or an imidazoquinoline diamine. As used herein, substituted imidazoquinoline amines specifically and expressly exclude 1-(2-methylpropyl)-1H-imidazo[4,5-c]quinolin-4-amine and 4-amino-α,α-dimethyl-2-ethoxymethyl-1H-imidazo[4,5-c]quinolin-1-ethanol.

In one embodiment, the IRM compound may be a sulfonamide substituted imidazoquinoline amine such as, for example, N-[4-(4-amino-2-ethyl-1H-imidazo[4,5-c]quinolin-1-yl)butyl]methanesulfonamide.

In another embodiment, the IRM compound may be a thiazoloquinoline amine such as, for example, 2-propylthiazolo[4,5-c]quinolin-4-amine.

Suitable IRM compounds also may include the purine derivatives, imidazoquinoline amide derivatives, benzimidazole derivatives, adenine derivatives, aminoalkyl glucosaminide phosphates, and oligonucleotide sequences described above.

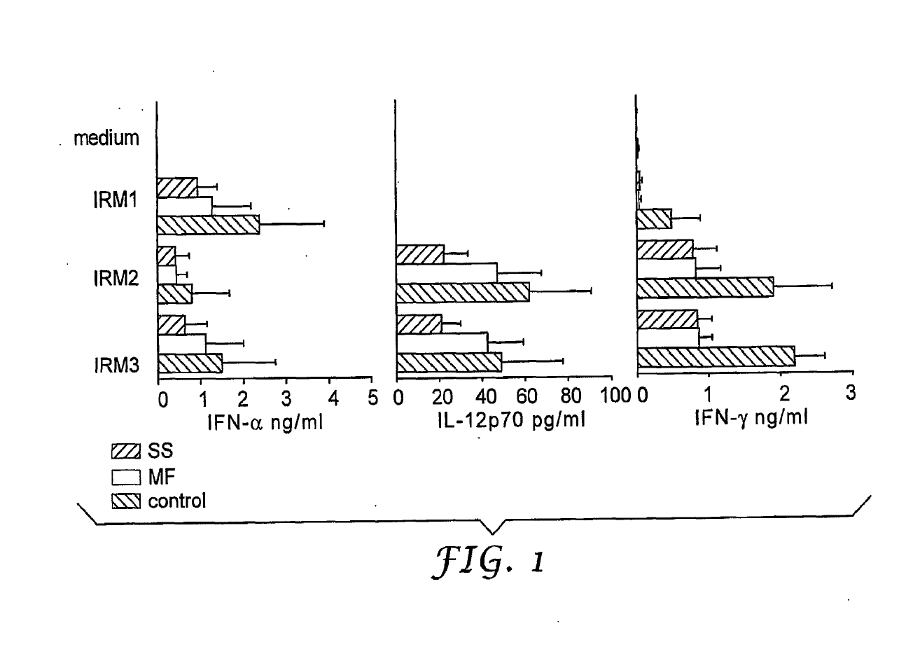

FIG. 1 illustrates that IRM compounds, particularly TLR8 agonists, were able to induce cytokine production from peripheral blood mononuclear cells (PBMCs) collected from CTCL patients, despite these patients having reduced numbers of dendritic cells among their PBMCs. PBMCs of CTCL patients and volunteers were stimulated to produce IFN-α by IRM2, an unexpected result.

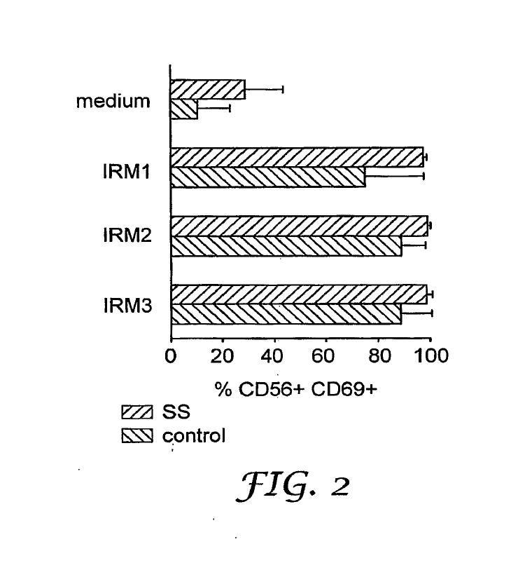

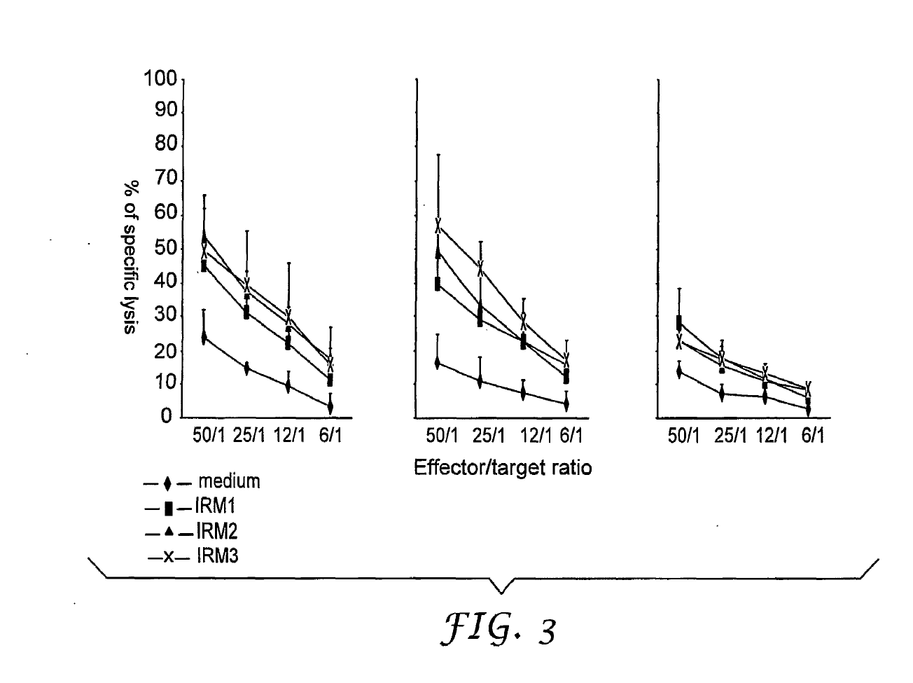

Additionally, stimulation with IRM compounds cause upregulation of CD69 expression on NK cells of CTCL patients (FIG. 2). Moreover, cytolytic activity of NK cells was also increased by all IRM compounds tested (FIG. 3). Cytolytic activity in the samples from Sezary syndrome patients was somewhat less than that observed in the samples from MF patients and healthy volunteers. However, the increase in cytolytic activity was quite marked, especially considering the reduction in peripheral blood NK cells typically observed in Sezary syndrome patients.

CTCL patients, particularly Sezary syndrome patients, are deficient in IL-12 production resulting, at least in part, from decreased numbers of myeloid dendritic cells, which are important IL-12 producers. IL-12 stimulates proliferation of NK cells and T cells, increases cytolytic activity of NK cells, and stimulates IFN-γ production, which in turn enhances production of IL-12 by DCs and monocytes.

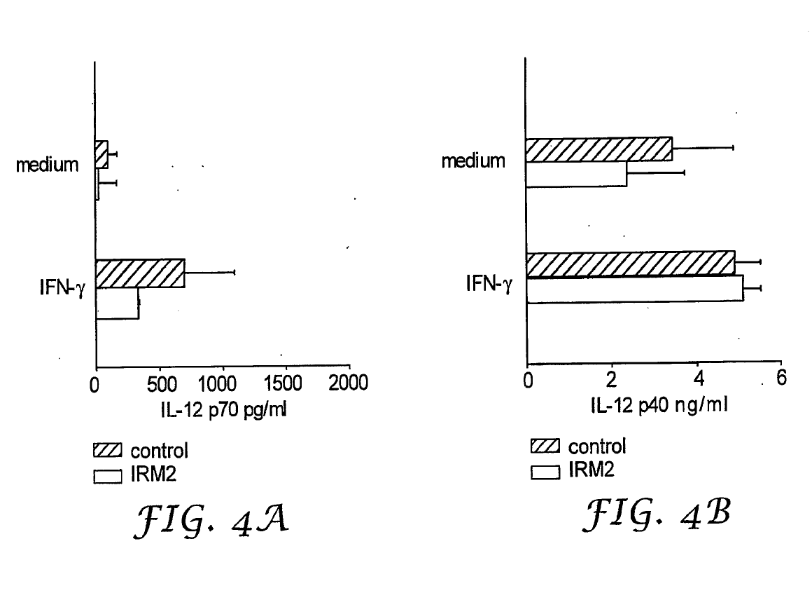

Pretreatment of PBMCs with a Type I interferon such as, for example, IFN-α or IFN-γ significantly increases the production of IL-12 by PBMCs stimulated with IRM compounds. In fact, IFN-γ priming results in levels of IL-12 production comparable with those of healthy volunteers subjected to the same treatment (FIG. 4). Thus, in certain embodiments, the methods of the invention can include a contacting a cell population a priming dose of a Type I interferon (e.g., IFN-α or IFN-γ) or administering to a patient a priming dose of a Type I interferon. The Type I interferon may be recombinantly-derived or naturally-occurring.

The IRM compound may be provided in any formulation suitable for contacting cells in vitro or administering to a subject. Suitable types of formulations are described, for example, in U.S. Pat. No. 5,238,944; U.S. Pat. No. 5,939,090; U.S. Pat. No. 6,245,776; European Patent No. EP 0 394 026; U.S. Patent Publication No. 2003/0199538; and International Patent Publication Nos. WO 2006/073940 and WO 2006/074045. The compound may be provided in any suitable form including but not limited to a solution, a suspension, an emulsion, or any form of mixture. The compound may be delivered in formulation with any pharmaceutically acceptable excipient, carrier, or vehicle. For example, the formulation may be delivered in a conventional topical dosage form such as, for example, a cream, an ointment, an aerosol formulation, a non-aerosol spray, a gel, a lotion, and the like. The formulation may further include one or more additives including but not limited to adjuvants, skin penetration enhancers, colorants, fragrances, flavorings, moisturizers, thickeners, and the like.

A formulation containing an IRM compound may be administered in any suitable manner such as, for example, non-parenterally or parenterally. As used herein, non-parenterally refers to administration through the digestive tract, including by oral ingestion. Parenterally refers to administration other than through the digestive tract such as, for example, intravenously, intramuscularly, transdermally, subcutaneously, transmucosally (e.g., by inhalation), or topically.

The composition of a formulation suitable for practicing the invention will vary according to factors known in the art including but not limited to the physical and chemical nature of the IRM compound, the nature of the carrier, the intended dosing regimen, the state of the subject's immune system (e.g., suppressed, compromised, stimulated), the method of administering the IRM compound, and the species to which the formulation is being administered. Accordingly, it is not practical to set forth generally the composition of a formulation effective for treating cutaneous T cell lymphoma for all possible applications. Those of ordinary skill in the art, however, can readily determine an appropriate formulation with due consideration of such factors.

In some embodiments, the methods of the present invention include administering IRM compound to a subject in a formulation of, for example, from about 0.0001% to about 20% (unless otherwise indicated, all percentages provided herein are weight/weight with respect to the total formulation) to the subject, although in some embodiments the IRM compound may be administered using a formulation that provides IRM compound in a concentration outside of this range. In certain embodiments, the method includes administering to a subject a formulation that includes from about 0.01% to about 1% IRM compound, for example, a formulation that includes about from about 0.1% to about 0.5% IRM compound.

An amount of an IRM compound effective for treating cutaneous T cell lymphoma is an amount sufficient to limit, reduce, ameliorate, or slow the progression or severity of at least one symptom or clinical sign of CTCL. The precise amount of IRM compound for treating cutaneous T cell lymphoma will vary according to factors known in the art including but not limited to the physical and chemical nature of the IRM compound, the nature of the carrier, the intended dosing regimen, the state of the subject's immune system (e.g., suppressed, compromised, stimulated), the method of administering the IRM compound, and the species to which the IRM compound is being administered. Accordingly, it is not practical to set forth generally the amount that constitutes an amount of IRM compound effective for treating cutaneous T cell lymphoma for all possible applications. Those of ordinary skill in the art, however, can readily determine the appropriate amount with due consideration of such factors.

In some embodiments, the methods of the present invention include administering sufficient IRM compound to provide a dose of, for example, from about 100 ng/kg to about 50 mg/kg to the subject, although in some embodiments the methods may be performed by administering IRM compound in a dose outside this range. In some of these embodiments, the method includes administering sufficient IRM compound to provide a dose of from about 10 μg/kg to about 5 mg/kg to the subject, for example, a dose of from about 100 μg/kg to about 1 mg/kg.

In some embodiments, the dose may be calculated using actual body weight obtained just prior to the beginning of the treatment course. For such dosages, body surface area (m²) may be calculated prior to the beginning of the treatment course using the Dubois method: m²=(wt kg^{0.425}×height cm^{0.725})×0.007184.

In such embodiments, methods of the present invention include administering sufficient IRM compound to provide a dose of, for example, from about 0.01 mg/m² to about 5.0 mg/m² to the patient, although in some embodiments the methods may be performed by administering IRM compound in a dose outside this range. In some of these embodiments, the method includes administering sufficient TLR agonist to provide a dose of from about 0.1 mg/m² to about 2.0 mg/m² to the patient.

The dosing regimen and duration of therapy may depend at least in part on many factors known in the art including but not limited to the physical and chemical nature of the IRM compound, the nature of the carrier, the amount of IRM being administered, the state of the subject's immune system (e.g., suppressed, compromised, stimulated), the method of administering the IRM compound, and the species to which the IRM compound is being administered. Accordingly it is not practical to set forth generally the dosing regimen and duration of therapy effective for treating cutaneous T cell lymphoma for all possible applications. Those of ordinary skill in the art, however, can readily determine an appropriate dosing regimen and therapy duration with due consideration of such factors.

In some embodiments of the invention, the IRM compound may be administered, for example, from a single administration to about once per day, although in some embodiments the methods of the present invention may be performed by administering the IRM compound at a frequency outside this range. In certain embodiments, the IRM compound may be administered from about once per month to about twice per week. In one particular embodiment, the IRM compound is administered twice per week.

In some embodiments, the IRM compound may be administered on an “as needed” basis, i.e., only when symptoms or clinical signs of cutaneous T cell lymphoma appear. In other embodiments, the IRM compound may be administered over a prescribed duration of time. Administration of the IRM compound may be continuous throughout a prescribed period of time or, alternatively, rest periods may be incorporated into the therapy period. The duration of therapy may be, for example, at least two weeks, at least four weeks, at least eight weeks, or at least twelve weeks. In one particular embodiment, the IRM compound may be administered twice per week for twelve weeks.

### EXAMPLES

The following examples have been selected merely to further illustrate features, advantages, and other details of the invention. It is to be expressly understood, however, that while the examples serve this purpose, the particular materials and amounts used as well as other conditions and details are not to be construed in a matter that would unduly limit the scope of this invention.

### IRM Compounds

The IRM compounds used in the examples are shown in Table 1.

TABLE 1

| Compound | Chemical Name | Reference |
|---|---|---|
| IRM1 | N-[4-(4-amino-2-ethyl-1H-imidazo[4,5-c]quinolin- | U.S. Pat. No. 6,677,349 |
| | 1-yl)butyl]methanesulfonamide | Example 236 |
| IRM2 | 2-propylthiazolo[4,5-c]quinolin-4-amine | U.S. Pat. No. 6,110,929 |
| | | Example 12 |
| IRM3 | 4-amino-α,α-dimethyl-2-ethoxymethyl-1H- | U.S. Pat. No. 5,389,640 |
| | imidazo[4,5-c]quinolin-1-ethanol | Example 99 |

### Example 1

Peripheral blood samples were obtained from mycosis fungoides (MF) patients and Sezary syndrome (SS) patients. Flow cytometric analysis of peripheral blood samples with assessment of the numbers of CD4⁺/CD26⁻/CD7⁻ cells was routinely used to quantify the numbers of circulating malignant T cells. Absence of circulating malignant T cells was verified by examination of one-micron sections of formalin-fixed peripheral blood buffy coats for lymphocytes with atypical ceribriform appearing nuclei. Patients were divided into three groups based on tumor load in their circulation as described in Wysocka et al., Blood (2002), 100:3287-3294. Those with between 5% and 19% circulating Sezary cells were defined as low tumor burden patients; those with between 20% and 50% circulating Sezary cells were defined as medium tumor burden patients; and those with greater than 50% circulating Sezary cells were defined as high tumor burden patients. All patients were receiving identical treatment consisting of extracorporeal photopheresis on approximately an every four-week schedule, as described in Rook et al., J. Invest. Dermatol. Symp. Proc. (1999), 4:85-90. Blood samples from age-matched healthy volunteers were used as controls.

Peripheral blood mononuclear cells (PBMCs) were collected from patients and control samples as described in Rook et al., J. Immunol. (1995), 154-1491-1498. Cells were cultured in RPMI 1640 (Life Technologies, Inc., Gaithersburg, Md.), supplemented with 10% fetal bovine serum (FBS), penicillin/streptomycin, and L-glutamine (Gibco-BRL, Grand Island, N.Y.). To induce immune responses in vitro, PBMCs were cultured in 24-well plates at a density of 2×10⁶/mL/well for 18-24 hours with IRM1, IRM2, or IRM3 at a final concentration of 10 μg/mL.

Cells were harvested for flow cytometric analysis and the remaining supernatants were collected for cytokine analysis.

Cell free supernatants were collected from low/medium burden Sezary syndrome patients (SS, n—8), mycosis fungoides patients (MF, n=4), and healthy volunteers (control, n=8). The supernatants were tested for the presence of IFN-α, IL-12p70, IL-12p40, and IFN-γ by standard ELISA, using antibody pairs from Endogen (IFN-α, Woburn, Mass., sensitivity 10 pg/mL) or R&D Systems, Inc. (IFN-γ, IL-12p70, IL-12p40, Minneapolis, Minn., sensitivity 10 pg/mL for IFN-γ and IL-12p70, 20 pg/mL for IL-12p40). Results are shown in FIG. 1.

### Example 2

Cells were harvested for flow cytometric analysis to determine intracellular expression of cellular markers by NK cells. Unless otherwise indicated, all antibodies were obtained from BD Biosciences, San Jose, Calif. To determine expression of CD69 by NK cells or T cells, PBMCs from Sezary syndrome patients (SS, n=8) and health volunteers (control, n=8) were stained with either (a) anti-CD3-PerCp, anti-CD56/CD16-APC, and anti-CD69-FITC, or (b) anti-CD4-APC, anti-CD8-PerCp, and antiCD69-FITC.

Cells were analyzed with a FACSCALIBUR using CELLQuest software (both from Becton Dickinson, San Jose, Calif.) at the Flow Cytometry and Cell Sorting Core, Abramson Cancer Center, University of Pennsylvania, Philadelphia, Pa. 150,000 events were collected to analyze dendritic cells and NK cells. Results are shown in FIG. 2.

### Example 3

PBMCs were stimulated for 24 hours with either IRM 1, IRM2, or IRM3 as described above. After incubation with IRM compound, the cells were harvested, washed with PBS (Gibco-BRL, Grand Island, N.Y.) and replated. Human lymphoblastoma K562 cells (ATCC, Rockville, Md., CCL# 243) were used as targets. A standard 4-hour Cr 5-release assay was performed as described in Rook et al., J. Immunol. (1995), 154:1491-1498. Results are shown in FIG. 3.

### Example 4

PBMCs from Sezary syndrome (SS) patients and healthy volunteers (control) were obtained as described in Example 1. Cells were stimulated with either medium or IFN-γ (10 ng/mL) for 24 hours. Cells were then washed twice with PBS and restimulated with IRM2 (10 μg/mL) for an additional 24 hours. IL-12 levels were measured in cell-free supernatants as described in Example 1. Results are shown in FIG. 4.

The complete disclosures of the patents, patent documents and publications cited herein are incorporated by reference in their entirety as if each were individually incorporated. In case of conflict, the present specification, including definitions, shall control.

Various modifications and alterations to this invention will become apparent to those skilled in the art without departing from the scope and spirit of this invention. Illustrative embodiments and examples are provided as examples only and are not intended to limit the scope of the present invention. The scope of the invention is limited only by the claims set forth as follows.

1. (canceled)

2. A method of increasing a cell-mediated immune response of a cell population that includes cells affected by cutaneous T cell lymphoma, the method comprising:
contacting the cell population with an IRM compound in an amount effective to increase at least one cell-mediated immune activity of the cell population, wherein the IRM compound is a substituted imidazoquinoline amine, a tetrahydroimidazoquinoline amine, an imidazopyridine amine, a 1,2-bridged imidazoquinoline amine, a 6,7-fused cycloalkylimidazopyridine amine, an imidazonaphthyridine amine, a tetrahydroimidazonaphthyridine amine, an oxazoloquinoline amine, a thiazoloquinoline amine, an oxazolopyridine amine, a thiazolopyridine amine, an oxazolonaphthyridine amine, a thiazolonaphthyridine amine, a pyrazolopyridine amine, a pyrazoloquinoline amine, a tetrahydropyrazoloquinoline amine, a pyrazolonaphthyridine amine, or a tetrahydropyrazolonaphthyridine amine.

3. A method of treating a patient with cutaneous T cell lymphoma, the method comprising:
administering to the patient an amount of a pharmaceutical composition comprising an IRM compound effective for ameliorating at least one symptom or clinical sign of cutaneous T cell lymphoma, wherein the IRM compound is other than 1-(2-methylpropyl)-1H-imidazo[4,5-c]quinolin-4-amine.

4. A method of treating a patient with cutaneous T cell lymphoma, the method comprising:
administering to the patient an amount of a pharmaceutical composition comprising an IRM compound effective for ameliorating at least one symptom or clinical sign of cutaneous T cell lymphoma, wherein the IRM compound is a substituted imidazoquinoline amine, a tetrahydroimidazoquinoline amine, an imidazopyridine amine, a 1,2-bridged imidazoquinoline amine, a 6,7-fused cycloalkylimidazopyridine amine, an imidazonaphthyridine amine, a tetrahydroimidazonaphthyridine amine, an oxazoloquinoline amine, a thiazoloquinoline amine, an oxazolopyridine amine, a thiazolopyridine amine, an oxazolonaphthyridine amine, a thiazolonaphthyridine amine, a pyrazolopyridine amine, a pyrazoloquinoline amine, a tetrahydropyrazoloquinoline amine, a pyrazolonaphthyridine amine, or a tetrahydropyrazolonaphthyridine amine.

5. The method of claim 2 wherein the cell-mediated cellular activity comprises production of a T_{H}1 cytokine.

6. The method of claim 5 wherein the T_{H}1 cytokine comprises IFN-α.

7. The method of claim 5 wherein the T_{H}1 cytokine comprises IL-12.

8. The method of claim 5 wherein the T_{H} 1 cytokine comprises IFN-γ.

9. The method of claim 2 wherein the cell population comprises CD4⁺/CD45RO⁺/CLA⁺/CCR4⁺ T lymphocytes.

10. The method of of claim 3 wherein the IRM compound comprises an agonist of at least one of TLR7 and TLR8.

11. The method of claim 10 wherein the IRM compound comprises a TLR8-selective compound.

12. The method of claim 10 wherein the amount of IRM compound is an amount effective to induce production of IFN-α.

13. The method of of claim 3 wherein the IRM compound is a sulfonamide substituted imidazoquinoline amine or a thiazoloquinoline amine.

14-19. (canceled)

20. The method of claim 3 further comprising
administering to the patient a priming dose of IFN-α or IFN-γ in an amount effective to increase IRM-induced IL-12 production in the patient compared to IRM-induced IL-12 production in the patient in the absence of the priming dose.

21-31. (canceled)
